(19) United States
(12) Patent Application Publication
(10) Pub. No.: US 2025/0107696 A1
(43) Pub. Date: Apr. 3, 2025

(54) ENDOSCOPE CONTAMINATION PREVENTION METHOD AND ENDOSCOPE STORAGE AND TRANSPORTATION DEVICES

(71) Applicant: OLYMPUS Winter & Ibe GmbH, Hamburg (DE)

(72) Inventor: Dennis AEHLIG, Hamburg (DE)

(73) Assignee: OLYMPUS Winter & Ibe GmbH, Hamburg (DE)

(21) Appl. No.: 18/812,809

(22) Filed: Aug. 22, 2024

Related U.S. Application Data
(60) Provisional application No. 63/541,358, filed on Sep. 29, 2023.

(51) Int. Cl. A61B 1/00

(52) U.S. Cl. CPC A61B 1/00142; A61B 1/00097; A61B 1/00135; A61B 1/00149

(57) ABSTRACT

The present disclosure is directed at an endoscope contamination prevention method and an endoscope storage and/or transportation device. The endoscope contamination prevention method comprises using blue light to prevent contamination of surfaces of a compartment of an endoscope storage and/or transportation device that can come into contact with an inserted endoscope, such blue light can have a wavelength between 400 nm and 470 nm, such as, 405 nm.

### CROSS-REFERENCE TO RELATED APPLICATION

The present application is based upon and claims the benefit of priority from U.S. Provisional Application No. 63/541,358 filed on Sep. 29, 2023, the entire contents of which is incorporated herein by reference.

### BACKGROUND

### Field

The present disclosure relates to an Endoscope contamination prevention method and to an endoscope storage and/or transportation device.

### Prior Art

Latest standards and local guidelines emphasize that it is of utmost importance to dry and store flexible endoscopes safely.

The most common method to reprocess flexible endoscopes is to submit them to a high-level disinfection (HLD) process. Afterwards the flexible endoscope is used immediately or needs to be dried and stored. The endoscopes are dried and stored in special devices, so called drying and storage cabinets. The flexible endoscope can remain in the drying and storage cabinet for longer periods that typically range from 1 to 4 weeks.

It is common that several endoscopes are stored within the same compartment of a drying and storage cabinet, which poses a risk of cross-or recontamination of the co-stored endoscope. Main sources of germs for recontamination are use errors, where, e.g., a dirty endoscope is introduced in the drying and storage cabinet, technical defects or regrowth of bacteria as the endoscopes are reprocessed with HLD methods and not sterilized, which results in a certain bioburden to remain. The risk of recontamination needs also exists in transport systems that are used to bring the flexible endoscope from a reprocessing area to a drying and storage cabinet or a patient, such as in mobile endoscopy trolleys with compartments used to transport endoscopes from a drying and storage cabinet to an operating room (OR) and back, or to and from reprocessing or disinfection stations.

The main method to avoid contamination of the endoscope during transport, drying and storage is to conduct manual cleaning and disinfection of the involved products on a regular basis. However, manual cleaning and disinfection of the surfaces that are in contact with the flexible endoscope is a time-consuming process which has at least the following disadvantages. It can entail significant cost of labor due to its labor intensity, the usage of chemicals poses a health risk to the personnel, such chemicals can be aggressive and deteriorate the material of the involved products, and use errors that lead to an insufficient cleaning and disinfection can occur, i.e., chemicals are not used correctly or not distributed over the entire surface that may come into contact with an endoscope.

### SUMMARY

An object to prevent cross-or recontamination of endoscopes after they have been cleaned and disinfected and before they are used again.

Such object can be solved by an endoscope contamination prevention method, the method comprising inserting at least one endoscope into a compartment of an endoscope storage and/or transportation device, the compartment having one or more internal surfaces that may or will come into contact with an inserted endoscope, closing at least one of the compartment and the endoscope storage and/or transportation device for storage and/or transportation of the at least one inserted endoscope, starting application of blue light to the internal surfaces that may be or are in contact with the at least one inserted endoscope, opening at least one of the compartment and the endoscope storage and/or transportation device for extraction of the at least one inserted endoscope, upon opening at least one of the compartment and the endoscope storage and/or transportation device for extraction of the at least one inserted endoscope, stopping application of blue light to the internal surfaces that may be or are in contact with the at least inserted endoscope, and extracting the at least inserted endoscope from the compartment.

The endoscope contamination prevention method exposes the surfaces of the products that are in contact with clean flexible endoscopes to light of a certain wavelength that deactivates and destroys microorganism, thus, rendering manual disinfection unnecessary.

Flexible endoscopes themselves are already disinfected using UV light. However, a disadvantage of UV light, in particular UVA or UVB light, is that it causes severe material deterioration over time, such as due to the induced cross linking of plastics leading to embrittlement. Furthermore, UV light is dangerous for the user which means that special precautions need to be taken in order to prevent emission of UV radiation.

In contrast to this, it is presently proposed to use blue light in the visible spectrum, such as having wavelengths of 400 nm to 470 nm, to disinfect surfaces that may come into contact with endoscopes. This wavelength is a lot less harmful and can be applied continuously within the drying and storage cabinet or endoscope transport system.

Blue light is very efficient in the destruction of germs (bacteria, viruses, fungi, and parasites), biofilm, and even multi-drug resistant organisms, as has been found out in clinical studies, such as Cabral, J. and Rodrigues A G, “blue light Disinfection in Hospital Infection Control: Advantages, Drawbacks, and Pitfalls”, Antibiotics (2019), 8, 58. For this solution, no exogeneous photosensitizer is required as endogenous photosensitizers of microorganisms are used. The effect of blue light can be increased when additional photosensitizers such as tetrapyrrol derivatives or synthetic analogues, such as meso-tetraarylporphyrins, phthalocyanines, porphycans, texaphyrins, and safirins are incorporated in the surface. However, blue light in comparison to other kinds of photodynamic disinfection methods does not require additional photosensitizers to be effective.

Light sources that emit blue light can be easily integrated in the chamber of the drying and storage cabinet. Thus, all parts of the device can be subjected to the disinfection. Furthermore, integration in door handles, touch displays, and other parts of the device that are in contact with the user is possible and beneficial because contamination will be transferred to and from these surfaces, which increases the risk of contamination of the flexible endoscope. Additionally, blue light prevents the recontamination of the flexible endoscopes that are dried, stored or transported.

The application of blue light for disinfection according to the above described method bears no extra costs or manufacturing steps for material coating, low maintenance requirements, low material stress compared to other light disinfection methods, is harmless for the user, is effective against a wide variety of pathogens and no resistance is formed by the microorganisms and useful for continuous disinfection of air and surfaces. This can result in improved safety against recontamination of reprocessed flexible endoscopes, benefits personnel safety and lowers overall costs.

In embodiments, the blue light can have a wavelength between 400 nm and 470 nm, such as, 405 nm. Blue light in this range of wavelengths can be effective in disinfecting endoscopes without the detrimental effects of ultraviolet light on materials and people.

At least one of the inserting and the extracting of the at least one endoscope into or from the compartment can be done one of manually and by automated means in embodiments, the automated means can comprise at least one of a robotic arm and an automated sled.

The starting and the stopping of the application of blue light may in embodiments be actuated manually, or automatically upon a sensor signal from one or more mechanical and/or optical sensors sensing the closing and opening of the compartment and/or the endoscope storage and/or transportation device, and/or the presence of at least one inserted endoscope. The illumination with blue light may in addition also be performed when a compartment is open. However, illuminating the compartment in a closed state will shield users from having to look into bright blue light.

In embodiments, closing at least one of the compartment and the endoscope storage and/or transportation device can comprise at least one of closing a door or sliding a compartment tray into the endoscope storage and/or transportation device.

Blue light may be applied one of continuously and intermittently, wherein in case of intermittent application of blue light durations of alternating application and non-application of blue light being set with predetermined values that have been proven to allow for satisfactory disinfection of the concerned surfaces for the purposes of preventing contamination of disinfected and cleaned endoscope. Such object can also be achieved with an endoscope contamination prevention method, the method comprising using blue light to prevent contamination of surfaces of a compartment of an endoscope storage and/or transportation device that may or will come into contact with an inserted endoscope, such blue light can have a wavelength between 400 nm and 470 nm, such as, 405 nm.

Furthermore, such object can be achieved with an endoscope storage and/or transportation device having at least one compartment for at least one endoscope to be inserted, the at least one compartment having one or more internal surfaces that may or will come into contact with an inserted endoscope, wherein the endoscope storage and/or transportation device having one or more blue light illumination sources, the blue light illumination sources being located and oriented to emit blue light onto one or more internal surfaces of the at least one compartment that may or will come into contact with an inserted endoscope. Being equipped with blue light illumination sources direct at the internal surfaces to be disinfected or kept from infection, the endoscope storage and/or transportation device can fulfil the same purpose and embodies the same features and advantages as the above-described method.

In embodiments, the device may be one of a drying and storage cabinet and a mobile endoscopy trolley. Other devices used for storing or transporting endoscopes or for cleaning and disinfecting endoscopes may be equipped in the same manner blue light illumination sources directed at surfaces that may come into contact with endoscopes.

The blue light emission sources in embodiments, can be equipped to emit light in with a wavelength or wavelengths between 400 nm and 470 nm, such as 405 nm, thus providing good disinfection properties while avoiding the drawbacks of UV lighting.

The endoscope storage and/or transportation device may in embodiments further comprise at least one of a door and a slideable compartment tray. In that case, the endoscope storage and/or transportation device may further comprise a switch connecting a power source with the blue light illumination sources, the switch being switchably connected with the door or the slideable compartment tray such as to turn on the blue light illumination sources when the door is closed or the compartment tray is slid into the endoscope storage and/or transportation device. The use of the switch can be such that blue light is only applied when the device is closed, so that the eyes of users are kept from being irritated by intense blue light.

In embodiments, the endoscope storage and/or transportation device can further comprise automation for inserting and/or extracting the at least one endoscope into or from the compartment, such as, at least one of a robotic arm and an automated sled.

The endoscope storage and/or transportation device may, in embodiments, further comprise one or more mechanical and/or optical sensors adapted and configured for sensing the closing and opening of the compartment and/or the endoscope storage and/or transportation device, and/or the presence of at least one inserted endoscope. In that case, the endoscope storage and/or transportation device may further comprise a controller adapted and configured for activating and deactivating blue light illumination of the at least one cabinet in reaction to sensor signals by the one or more mechanical and/or optical sensors.

In further embodiments, the endoscope storage and/or transportation device can further comprise a controller adapted and configured to control the application of blue light to being done one of continuously and intermittently, wherein in case of intermittent application of blue light durations of alternating application and non-application of blue light being set with predetermined values. An intermittent application of light may reduce the power requirements for the disinfection by blue light. The controller may be the same controller that is adapted are configured for activating and deactivating the blue light illumination in reaction to sensor signals in the previously described embodiment, or a separate controller.

The endoscope storage and/or transportation device may in embodiments further comprise additional photosensitizers incorporated into at least one internal surface that may or will come into contact with an inserted endoscope, such as, at least one of tetrapyrrol derivatives and synthetic analogues, such as at least one of meso-tetraarylporphyrins, phthalocyanines, porphycans, texaphyrins, and safirins.

### BRIEF DESCRIPTION OF THE DRAWINGS

Further features will become evident from the description of embodiments, together with the claims and the appended drawings. Embodiments can fulfill individual features or a combination of several features.

The embodiments described below, without restricting the general intent of the invention, based on exemplary embodiments, wherein reference is made expressly to the drawings with regard to the disclosure of all details that are not explained in greater detail in the text.

In the drawings:

FIG. 1 illustrates schematically a typical endoscope use and care cycle,

FIG. 2 illustrates schematically an embodiment of a drying and storage cabinet, and

FIG. 3 illustrates schematically an embodiment of an endoscope contamination prevention method.

In the drawings, the same or similar types of elements or respectively corresponding parts are provided with the same reference numbers in order to prevent the item from needing to be reintroduced.

### DETAILED DESCRIPTION

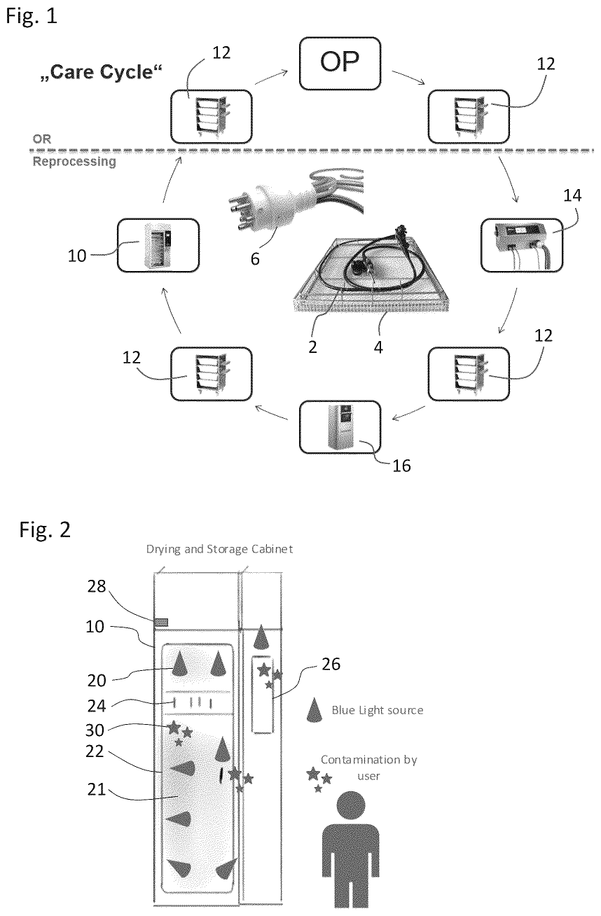

FIG. 1 illustrates schematically a typical endoscope use and care cycle. In the center, and endoscope 2 is positioned in a tray 4 typically used for disinfecting and reprocessing endoscopes. Furthermore, cables 6 are illustrated in the center of the care cycle. The care cycle itself is a closed cycle divided by a dashed line into a reprocessing part and an operating room (OR) part. Starting in the operating room parts, endoscope 2 may be transported by means of a mobile endoscopy trolley 12 having one or several trays or compartments into the operating room to be used in an operation OP, which may be a surgical or diagnostic procedure.

After completion of the operation OP, the used endoscope is put back into a compartment of mobile endoscopy trolley 12 to be transported out of the operating room OR and towards a first station of reprocessing, in this case a pre-cleaning station 14, such as the EPW 100 S type system by Steelco, wherein semi-automated pre-cleaning of the flexible endoscopes is conducted prior to the automatic reprocessing in a washer-disinfector.

By means of mobile endoscopy trolley 12 the used endoscope is then transported to reprocessing unit 16 where it is thoroughly cleaned and disinfected, to be transported by means of mobile endoscopy trolley 12 again to a drying and storage cabinet 10. Therein, the endoscope is dried and stored until its next use, to be extracted and transported into the operating room again.

FIG. 2 illustrates schematically an embodiment of a drying and storage cabinet 10 according to the present disclosure. The drying and storage cabinet 10 comprises a compartment 21 for storage of reprocessed endoscopes 2. The compartment 21 has inner surfaces 22 with which the endoscopes 2 stored inside the compartment 21 may come into contact. In order to prevent contamination of the endoscope 2, which contamination may be contamination 30 introduced by a user, blue light illumination sources 20 are located inside the compartment 21 such as to illuminate the inner surfaces 22 with intense blue light of wavelengths between 400nm and 470 nm. The wavelength may be a narrower range, for example centered around or located at 405 nm. Instead of a multiple of blue light illumination sources 20, one or two light illumination sources 20 may be employed for the most important in the surfaces 22.

Blue light illumination sources 20 may be chosen from available light sources such as blue LEDs, laser sources or incandescent sources, such as halogen lamps designed to emit high intensity blue light with or without blue light filters. Such blue light sources can, for example, be obtained from Heraeus Noblelight out of the BlueLight® product family or other sources.

A compartment 21 of the drying and storage cabinet 10 may be an enclosure into which the endoscopes are hung or a tray 4 such as displayed FIG. 1, or a tray of a mobile endoscopy trolley 12. The inner surfaces 22 may be surfaces of such trays or enclosures, but also surfaces of hooks inside an enclosure onto which an endoscope 2 may be hung.

The drying and storage cabinet may have mechanical and optical sensors 24 and or a switch 28 which may detect the opening and closing of the compartment 21 and thereby control the activation and deactivation of blue light illumination by the blue light illumination sources 20. An example would be a switch 28 that may open and close the electrical pathway of power for the blue light illumination sources 20. The switch 28 and/or the sensor or sensors 24 may also generate electrical signals that are fed into a controller 26 that controls blue light illumination sources 20 and activates illumination when the signal from the switch 28 or sensor 24 indicates that the compartment 21 is closed and, possibly, that an endoscope is present inside the compartment 21.

The compartment may be opened and closed by means of a door. Alternatively, in case of slidable trays, the opening and closing of the compartment may consist of sliding a tray into a opened or closed position, for example inside the mobile endoscopy trolley 12 which may be equipped with its own blue light illumination sources 20 and its own power supply and switches or controllers.

Controller 26 may also be configured to activate blue light illumination intermittently to conserve power, which may be especially advantageous in the case of mobile endoscopy trolley's 12 having their own power supply.

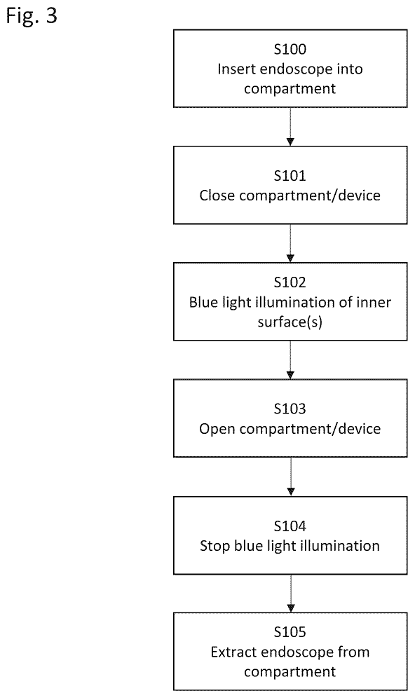

FIG. 3 illustrates schematically an embodiment of an endoscope contamination prevention method containing steps S100 to S105. In step S100, an endoscope 2 is inserted into a compartment 21 of a mobile endoscopy trolley 12 or a drying and storage cabinet 10. The compartment 21 and/or mobile endoscopy trolley 12 or drying and storage cabinet 10 is closed in step S101. In step S102, blue light illumination of the inner surfaces 22 of compartment 21 is activated in order to get rid of possible contamination on services that may come into contact with an endoscope 2. The blue light illumination may be applied continuously or intermittently in accordance with the requirements in each case, as long as efficient prevention of contamination of endoscopes 2 in the compartment 21 is assured.

Before a diagnostic or surgical procedure, the compartment 21 or device 10, 12 is opened in step S103, blue light illumination is stopped (step S104) and the endoscope 2 needed for the procedure is extracted from compartment 21 (step S105). The process is repeated after endoscope 2 has been used in the operating room and reprocessed and disinfected after use.

In addition, blue light illumination may also be applied to the inner surfaces 22 of compartment 21 in the absence of endoscopes 2 as a further means of preventing contamination later on. Furthermore, it is envisioned that blue light illumination may be active even when a compartment 21 is open. However, in that case either the intensity of the blue light illumination should be reduced in order to prevent users vision from being affected by the high intensity blue light illumination or physical means of blocking the blue light from reaching the user should be installed.

The most common use would be to apply blue light in order to keep a disinfected endoscope in that state, i.e., switching on the blue light when an endoscope is present. The chamber is then usually closed in order to maintain an overpressure in the chamber.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE SIGNS

2 endoscope
4 tray
6 cables
10 drying and storage cabinet
12 mobile endoscopy trolley
14 pre-cleaning station
16 reprocessor
20 blue light illumination source
21 compartment
22 inner surface
24 mechanical and optical sensors
26 controller
28 switch
30 contamination

What is claimed is:

1. An endoscope contamination prevention method, the method comprising:
inserting at least one endoscope into a compartment of an endoscope storage and/or transportation device, the compartment having one or more internal surfaces that can come into contact with an inserted endoscope,
subsequent to the inserting, closing at least one of the compartment and the endoscope storage and/or transportation device for storage and/or transportation of the at least one inserted endoscope,
subsequent to the closing, starting application of blue light to the internal surfaces,
subsequent to the starting, opening the at least one of the compartment and the endoscope storage and/or transportation device for extraction of the at least one inserted endoscope,
upon opening at least one of the compartment and the endoscope storage and/or transportation device for extraction of the at least one inserted endoscope, stopping application of blue light to the internal surfaces, and
extracting the at least one endoscope from the compartment.

2. The endoscope contamination prevention method according to claim 1, wherein the blue light having a wavelength between 400 nm and 470 nm.

3. The endoscope contamination prevention method according to claim 2, wherein the blue light having a wavelength of 405 nm.

4. The endoscope contamination prevention method according to claim 1, wherein at least one of the inserting and the extracting of the at least one endoscope into or from the compartment being done one of manually and by an automated device.

5. The endoscope contamination prevention method according to claim 4, wherein the automated device comprising at least one of a robotic arm and an automated sled.

6. The endoscope contamination prevention method according to claim 1, wherein the starting and the stopping of the application of blue light being actuated one of manually and automatically upon a sensor signal from one or more mechanical and/or optical sensors sensing the closing and opening of the compartment and/or the endoscope storage and/or transportation device, and/or the presence of at least one inserted endoscope.

7. The endoscope contamination prevention method according to claim 1, wherein the closing at least one of the compartment and the endoscope storage and/or transportation device comprises at least one of closing a door or sliding a compartment tray into the endoscope storage and/or transportation device.

8. The endoscope contamination prevention method according to claim 1, wherein the application of blue light is applied one of continuously and intermittently.

9. The endoscope contamination prevention method according to claim 8, wherein where the application of the blue light is intermittent, application of blue light durations of alternating application and non-application of blue light being set with predetermined values.

10. An endoscope contamination prevention method, the method comprising using blue light to prevent contamination of surfaces of a compartment of an endoscope storage and/or transportation device that can come into contact with an inserted endoscope.

11. The endoscope contamination prevention method of claim 10, wherein the blue light having a wavelength between 400 nm and 470 nm.

12. The endoscope contamination prevention method according to claim 11, wherein the blue light having a wavelength of 405 nm.

13. An endoscope storage and/or transportation device comprising:
at least one compartment for at least one endoscope to be inserted, the at least one compartment having one or more internal surfaces that can come into contact with an inserted endoscope; and
one or more blue light illumination sources disposed in the at least one compartment, the one or more blue light illumination sources being located and oriented to emit blue light onto one or more internal surfaces of the at least one compartment that can come into contact with an inserted endoscope.

14. The endoscope storage and/or transportation device of claim 13, the device being one of a drying and storage cabinet and a mobile endoscopy trolley.

15. The endoscope storage and/or transportation device of claim 13, wherein the one or more blue light emission sources being configured to emit light with a wavelength between 400 nm and 470 nm.

16. The endoscope storage and/or transportation device of claim 15, wherein the blue light emission sources being configured to emit light with a wavelength of 405 nm.

17. The endoscope storage and/or transportation device of claim 13, further comprising at least one of a door and a slideable compartment tray.

18. The endoscope storage and/or transportation device of claim 17, further comprising a switch connecting a power source with the one or more blue light illumination sources, the switch being switchably connected with the door or the slideable compartment tray such as to turn on the one or more blue light illumination sources when the door is closed or the compartment tray is slid into the endoscope storage and/or transportation device.

19. The endoscope storage and/or transportation device of claim 13, further comprising automated means for inserting and/or extracting the at least one endoscope into or from the compartment.

20. The endoscope storage and/or transportation device of claim 19, wherein the automated means comprises at least one of a robotic arm and an automated sled.

21. The endoscope storage and/or transportation device of claim 13, further comprising one or more mechanical and/or optical sensors configured for sensing the closing and opening of the compartment and/or the endoscope storage and/or transportation device, and/or the presence of at least one inserted endoscope.

22. The endoscope storage and/or transportation device of claim 21, further comprising a controller configured for activating and deactivating blue light illumination of the at least one cabinet in reaction to sensor signals by the one or more mechanical and/or optical sensors.

23. The endoscope storage and/or transportation device of claim 13, further comprising a controller configured to control the application of blue light to being done one of continuously and intermittently.

24. The endoscope storage and/or transportation device of claim 23, wherein where the blue light is applied intermittently, intermittent application of blue light durations of alternating application and non-application of blue light being set with predetermined values.

25. The endoscope storage and/or transportation device of claim 13, further comprising one or more photosensitizers incorporated into at least one internal surface that can come into contact with an inserted endoscope.

26. The endoscope storage and/or transportation device of claim 25, wherein the one or more photosensitizers are selected from a groups consisting of tetrapyrrol derivatives and synthetic analogues.

27. The endoscope storage and/or transportation device of claim 26, wherein the tetrapyrrol derivatives and synthetic analogues comprise at least one of meso-tetraarylporphyrins, phthalocyanines, porphycans, texaphyrins, and safirins.
